(19) United States
(12) Patent Application Publication
(10) Pub. No.: US 2024/0069103 A1
(43) Pub. Date: Feb. 29, 2024

(54) METHOD, SYSTEM AND STORAGE MEDIUM FOR SIMULATING DATA SET BASED ON ELECTROCHEMICAL MODEL

(71) Applicant: SHANGHAI MAKESENS ENERGY STORAGE TECHNOLOGY CO., LTD., Shanghai (CN)

(72) Inventors: Pingchao Hao, Shanghai (CN); Zhou Yang, Shanghai (CN); Zhimin Zhou, Shanghai (CN); Enhai Zhao, Shanghai (CN); Xiao Yan, Shanghai (CN)

(21) Appl. No.: 18/238,571

(22) Filed: Aug. 28, 2023

(30) Foreign Application Priority Data
Aug. 29, 2022 (CN) 202211041286.9

(51) Int. Cl. G01R 31/367; G06F 30/20

(52) U.S. Cl. CPC G01R 31/367; G06F 30/20

(57) ABSTRACT

The invention discloses a method, a system, and a storage medium for simulating a data set based on an electrochemical model. The method includes the following steps: setting geometric parameters and electrochemical parameters of a lithium battery; generating a volume average concentration of positive lithium ions and a volume average concentration of negative lithium ions under a preset state of charge through a simulation of the electrochemical model; calculating a capacity of the lithium battery through the simulation of the electrochemical model; after adjusting a current state of charge of the lithium battery, inputting a simulated operating current to acquire a simulated data set. The invention can generate operating data of a virtual battery by adjusting lithium battery electrochemical model parameters, facilitating management by a BMS system of battery operating states based on an electrochemical model of a virtual battery.

### CROSS-REFERENCE TO RELATED PATENT APPLICATION

This application claims priority to and the benefit of Chinese Patent Application No. 202211041286.9, filed Aug. 29, 2022, which is incorporated herein in its entirety by reference.

### FIELD OF THE INVENTION

The invention relates generally to the field of electrochemical model technology, and more particularly to a method, a system, and a storage medium for simulating a data set based on an electrochemical model.

### BACKGROUND OF THE INVENTION

In recent years, with the increased fossil fuel energy crisis and environmental issues, new energy industries represented by photovoltaics, wind energy, tidal energy, and biomass energy are rapidly developing. Due to the instability of power generation quality in new energy generation systems, energy storage systems need to be introduced to standardize the electrical energy parameters. Lithium ion battery energy storage systems have been widely used in the new energy field due to their energy storage stability.

Currently, to ensure the safety and reliability of lithium ion batteries in long-term use, a Battery Management System (BMS) consisting of software and hardware is needed to manage the working status of lithium ion batteries. Existing widely used BMS are developed based on Equivalent Circuit Models (ECM), but due to the limited predictive capabilities of ECMs, battery operation strategies are mostly designed based on simple safety constraints such as charge cut-off voltage, discharge cut-off voltage, maximum current, and the like. However, the terminal voltage of the battery cannot fully reflect the internal state of the battery, especially under high currents, which can greatly increase or decrease the terminal voltage of the battery during the charging and discharging processes due to a large overpotential.

With the advancement of hardware computational capabilities, a new type of more intelligent and advanced BMS based on Electrochemical Models (EM) is gradually becoming the direction of BMS improvement. EM can effectively reflect internal battery states such as positive and negative lithium ion concentration distribution, potential distribution, overpotential, etc., which significantly enhances ability of the BMS to manage the working status of lithium batteries. However, due to the involvement of a large number of coupled partial differential equations and numerous physical parameters in electrochemical models, the practical application of EM models is limited.

Therefore, there is currently a need for a data set simulation method based on an electrochemical model, which generates operational of a virtual battery by adjusting parameters of an electrochemical model of a lithium battery. This facilitates the management of the operational state of a battery by BMS systems based on an electrochemical model, using the generated operational data of the virtual battery.

### SUMMARY OF THE INVENTION

In order to address the technical problems of the limitations of the electrochemical model in practical applications due to its involvement of a large number of coupled partial differential equations and numerous physical parameters, the invention provides a method, a system and a storage medium for simulating a data set based on an electrochemical model, as set forth below.

The invention provides a method for simulating a data set based on an electrochemical model, comprising the following steps:
setting the geometric parameters and electrochemical parameters of the lithium battery;
generating, according to the geometric parameters and the electrochemical parameters, a volume average concentration of positive lithium ions and a volume average concentration of negative lithium ions under a preset state of charge through a simulation of the electrochemical model;
calculating, according to the volume average concentration of positive lithium ions and the volume average concentration of negative lithium ions under the preset state of charge, a capacity of the lithium battery through the simulation of the electrochemical model; and
after adjusting a current state of charge of the lithium battery, inputting a simulated operating current to acquire a simulated data set.

The method for simulating a data set based on an electrochemical model provided by this invention, through the steps of adjusting geometric parameters and electrochemical parameters of the lithium battery in the electrochemical model, standardizing parameters, calibrating capacity and adjusting the current state of charge of the lithium battery, obtains simulated data sets under different operating conditions based on the same electrochemical model, reducing the difficulty in obtaining the simulated data of the electrochemical model.

In some embodiments, the setting the geometric parameters and the electrochemical parameters of the lithium battery comprises:
setting the geometric parameters of the lithium battery, wherein the geometric parameters are battery structural parameters and are not influenced by an electrochemical reaction process after the lithium battery is manufactured; and
setting the electrochemical parameters according to adjustable requirements, wherein the electrochemical parameters are reaction coefficients in an electrochemical reaction of the battery and change along with progression of the electrochemical reaction process.

In some embodiments, the generating the volume average concentration of positive lithium ions and the volume average concentration of negative lithium ions under the preset state of charge through the simulation of the electrochemical model comprises:
inputting the geometric parameters and the electrochemical parameters into the electrochemical model; and
simulating, according to the electrochemical model, a standard charging process of the lithium battery under the preset state of charge, and calculating the volume average concentration of positive lithium ions and the volume average concentration of negative lithium ions.

In some embodiments, the calculating the capacity of the lithium battery through the simulation of the electrochemical model comprises:
inputting the volume average concentration of positive lithium ions and the volume average concentration of negative lithium ions into the electrochemical model; and
simulating, according to the electrochemical model, a standard discharge process of the lithium battery under the preset state of charge, and calculating the capacity of the lithium battery.

In some embodiments, the after adjusting the current state of charge of the lithium battery, inputting the simulated operating current and obtaining the simulated data set comprises:
changing the current state of charge of the lithium battery by adjusting an initial concentration of the positive lithium ions and an initial concentration of the negative lithium ions in the electrochemical model; and
inputting the simulated operating current into the electrochemical model to obtain the simulated data set output by the electrochemical model.

In some embodiments, the calculating the capacity of the lithium battery through the simulation of the electrochemical model further comprises:
after adjusting the current state of charge and a reaction temperature of the lithium battery, inputting the simulated operating current to obtain the simulated data set.

The method for simulating a data set based on an electrochemical model provided by this invention enhances the richness of the simulated data set by allowing the simulated temperature to be set, enabling acquisition of simulated data output by the battery cell under different SOCs and different temperatures.

In some embodiments, the simulated data set includes an OCV-SOC curve of the lithium battery.

In some embodiments, after the obtaining the simulated data set, the method further comprises:
inputting the simulated data set of the geometric parameters, the electrochemical parameters and labeled data tags into a preset neural network model, training and generating a battery data simulation model, wherein the data tags include at least one parameter of the geometric parameters, the electrochemical parameters and the capacity of the lithium battery.

The method for simulating a data set based on an electrochemical model provided by this invention simplifies the process of generating an electrochemical model simulated data set by training a battery data simulation model, an improvement leading to increased efficiency in generating electrochemical model simulated data sets.

In some embodiments, according to another aspect of the present invention, there is also provided a system for simulating a data set based on an electrochemical model, comprising:
a setting module, configured to set the geometric parameters and the electrochemical parameters of a lithium battery;
a generation module connected to the setting module, configured to generate, according to the geometric parameters and the electrochemical parameters, a volume average concentration of positive lithium ions and a volume average concentration of negative lithium ions under a preset state of charge through a simulation of the electrochemical model;
a calculation module respectively connected to the setting module and the generation module, configured to calculate, according to the volume average concentration of positive lithium ions and the volume average concentration of negative lithium ions under the preset state of charge, a capacity of the lithium battery through the simulation of the electrochemical model; and
an acquisition module respectively connected to the calculation module and the generation module, configured to, after adjusting a current state of charge of the lithium battery, input a simulated operating current to acquire a simulated data set.

In some embodiments, according to another aspect of the present invention, there is also provided a storage medium on which at least one instruction is stored, wherein the at least one instruction is loaded and executed by a processor to implement operations of the method for simulating a data set based on an electrochemical model as described above.

The invention provides a method, a system, and a storage medium for simulating a data set based on an electrochemical model, which at least comprise the following technical effects:
(1) By adjusting geometric parameters and electrochemical parameters of the lithium battery in the electrochemical model, standardizing parameters, calibrating capacity, and adjusting the current state of charge of the lithium battery, the method enables simulated data sets under different operating conditions to be obtained according to the same electrochemical model, reducing the difficulty in obtaining the simulated data of the electrochemical model;
(2) By allowing the simulated temperature to be set, the method enhances the richness of the simulated data set, enabling simulated data output by the battery cell under different SOCs and different temperatures to be obtained;
(3) By training a battery data simulation model, the method simplifies the process of generating simulated data sets based on electrochemical models, thereby increasing the efficiency of generating such data sets.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 is a flowchart of a method for simulating a data set based on an electrochemical model according to embodiments of the present invention;

FIG. 2 is a flowchart of the generation of a volume average concentration of positive lithium ions and a volume average concentration of negative lithium ions under a preset state of charge in a method for simulating a data set based on an electrochemical model according to embodiments of the present invention;

FIG. 3 is a flowchart of the calculation of a capacity of the lithium battery in a method for simulating a data set based on an electrochemical model according to embodiments of the present invention;

FIG. 4 is a flowchart of the adjustment of the current state of charge of a lithium battery in a method for simulating a data set based on an electrochemical model according to embodiments of the present invention;

FIG. 5 is a flowchart of the simultaneous adjustment of the current state of charge and the reaction temperature of a lithium battery in a method for simulating a data set based on an electrochemical model according to embodiments of the present invention;

FIG. 6 is another flowchart of a method for simulating a data set based on an electrochemical model according to embodiments of the present invention;

FIG. 7 is a diagram of a system for simulating a data set based on an electrochemical model according to embodiments of the present invention.

Reference numbers in the figures: a setting module—10, a generation module—20, a calculation module—30 and an acquisition module—40.

### DETAILED DESCRIPTION OF THE INVENTION

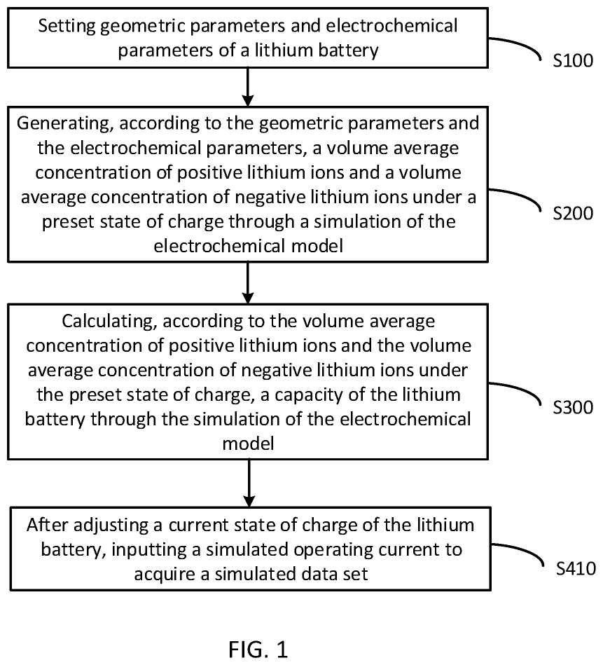

Embodiments of the invention are described below through specific examples in conjunction with the accompanying drawings in FIGS. 1-7, and those skilled in the art can easily understand other advantages and effects of the invention from the content disclosed in this specification. The invention can also be implemented or applied through other different specific implementations, and various modifications or changes can be made to the details in this specification according to different viewpoints and applications without departing from the spirit of the invention. It should be noted that, in the case of no conflict, the following embodiments and features in the embodiments can be combined with each other.

It should be noted that the drawings provided in the following embodiments are merely illustrative in nature and serve to explain the principles of the invention, and are in no way intended to limit the invention, its application, or uses. Only the components related to the invention are shown in the drawings rather than the number, shape and size of the components in actual implementations. For components with the same structure or function in some figures, only one of them is schematically shown, or only one of them is marked. They do not represent the actual structure of the product. Dimensional drawing, the type, quantity and proportion of each component can be changed arbitrarily in its actual implementations. More complicated component layouts may also become apparent in view of the drawings, the specification, and the following claims.

The terminology used herein is for the purpose of describing particular embodiments only and is not intended to be limiting of the invention. As used herein, “a” not only means “only one,” but also means “more than one.” The term “and/or” used in the description of the present application and the appended claims refers to any combination and all possible combinations of one or more of the associated listed items, and includes these combinations. The terms “first,” “second,” etc. are only used for distinguishing descriptions, and should not be construed as indicating or implying relative importance.

It should be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

In order to more clearly illustrate the embodiments of the invention or the technical solutions in the prior art, the following description will explain the specific embodiments of the invention with reference to the accompanying drawings. It is evident that the drawings in the following description are only examples of the invention, from which other drawings and other embodiments can be obtained by a person skilled in the art without inventive effort.

One embodiment of the present invention, as shown in FIG. 1, provides a method for simulating a data set based on an electrochemical model, comprising the following steps.

S100, setting geometric parameters and electrochemical parameters of the lithium battery.

Specifically, by altering relevant parameters within an electrochemical model of the lithium battery, a plurality of operating condition data sets can be generated through simulation of the operating condition of the battery in the electrochemical model. For example, in setting geometric parameters, at least one of the parameters of surface area of the active material, thickness of the Ln negative electrode active material, thickness of the Lp positive electrode active material, and thickness of the Ls separator can be set, and in setting electrochemical parameters, at least one of the parameters of volume fraction of the active material, thickness of the SEI film, and solid phase conductivity can be set.

S200, generating, according to the geometric parameters and the electrochemical parameters, a volume average concentration of positive lithium ions and a volume average concentration of negative lithium ions under a preset state of charge through a simulation of the electrochemical model.

Specifically, when modifying the electrochemical parameters, the volume average concentration of positive lithium ions and the volume average concentration of negative lithium ions of the lithium battery will also change accordingly. Therefore, after adjusting the geometric parameters and the electrochemical parameters, the parameters of the electrochemical model need to be standardized to obtain the volume average concentration of positive lithium ions and the volume average concentration of negative lithium ions under the preset state of charge.

S300, calculating the capacity of the lithium battery through electrochemical model simulation according to the volume average concentration of the positive lithium ions and the volume average concentration of the negative lithium ions in the preset charge state.

Specifically, after modifying the electrochemical model parameters, the capacity of the virtual lithium battery will change, so after obtaining the volume average concentration of positive lithium ions and the volume average concentration of negative lithium ions under the preset state of charge, simulation of the electrochemical model should be performed to obtain the capacity of the lithium battery after the model parameters are modified.

S410, after adjusting the current state of charge of the lithium battery, inputting a simulated operating current to acquire a simulated data set.

Specifically, to ensure comprehensive battery simulation data, simulation data for the virtual lithium battery across different states of charge are obtained, serving as starting points for performing operating condition simulations.

According to the method for simulating a data set based on an electrochemical model provided by this embodiment, simulated data sets under different operating conditions are obtained based on the same electrochemical model through the steps of adjusting geometric parameters and electrochemical parameters of the lithium battery in the electrochemical model, standardizing parameters, calibrating capacity and adjusting the current state of charge of the lithium battery, reducing the difficulty in obtaining the simulated data of the electrochemical model.

In an embodiment, in the execution process of step S100, geometric parameters of the lithium battery are set, the geometric parameters being structural parameters of the battery that are not affected by the electrochemical reaction process after manufacture of the battery, and electrochemical parameters are set according to adjustable requirements, the electrochemical parameters being reaction coefficients in the electrochemical reaction of the battery that change along with progression of the electrochemical reaction process.

In the implementation process of the disclosed technical scheme, different electrochemical models, such as an AMESim electrochemical model, a P2D electrochemical model, a P2D thermally coupled electrochemical model, and the like, may be adopted. When adopting different electrochemical models, in executing step S100 to set geometric parameters and electrochemical parameters of the lithium battery, differing model parameters need to be modified according to the model parameters. For example, when the P2D thermally coupled electrochemical model is adopted, at least one parameter of the following geometric parameters needs to be set: electrode effective area, positive electrode thickness, negative electrode thickness, separator thickness, positive electrode liquid volume fraction, positive electrode active material volume fraction, negative electrode liquid volume fraction, negative electrode active material volume fraction, separator liquid volume fraction, single cell capacity, positive electrode lithium intercalation amount at 0% SOC, negative electrode lithium intercalation amount at 0% SOC, positive electrode lithium ion maximum concentration, a negative electrode lithium ion maximum concentration, electrolyte lithium ion concentration, minimum electrolyte lithium ion concentration, lithium ion migration coefficient, positive electrode particle radius, negative electrode particle radius, SEI film reference potential, SEI film molar mass, SEI film density, SEI film porosity, and the like; and at least one parameter of the following electrochemical parameters needs to be set: active material volume fraction, SEI film thickness, solid phase conductivity, positive electrode solid phase diffusion coefficient, negative electrode solid phase coefficient, liquid phase diffusion coefficient, SEI film conductivity, SEI film surface concentration, positive electrode reaction rate coefficient, negative electrode reaction rate coefficient, and the like. The aforementioned parameters only represent a subset of the geometric parameters and the electrochemical parameters when utilizing a P2D thermally coupled electrochemical model, and their selection does not limit the scope of this scheme.

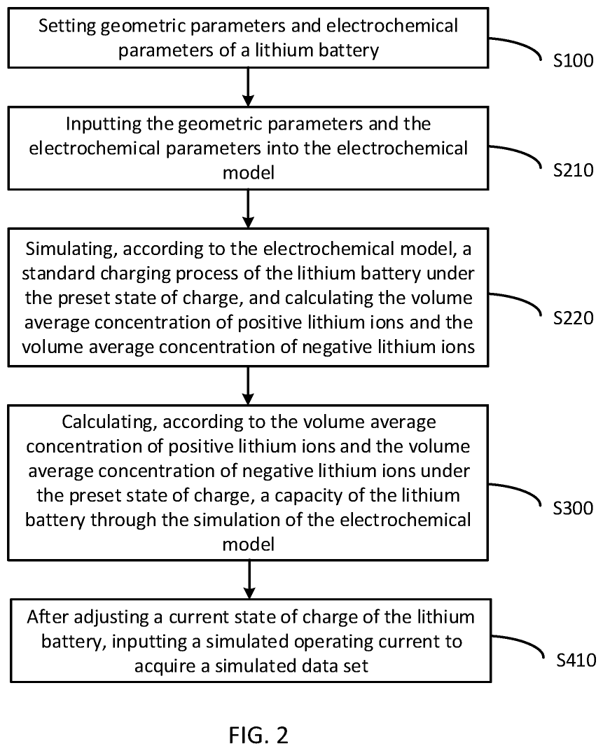

In an embodiment, as shown in FIG. 2, step S200 involves generating, according to the geometric parameters and the electrochemical parameters, a volume average concentration of positive lithium ions and a volume average concentration of negative lithium ions under a preset state of charge through a simulation of the electrochemical model, including:

S210, inputting the geometric parameters and the electrochemical parameters into the electrochemical model.

S220, simulating, according to the electrochemical model, a standard charging process of the lithium battery under the preset state of charge, and calculating the volume average concentration of positive lithium ions and the volume average concentration of negative lithium ions.

Illustratively, a standard charging process of a lithium battery at SOC=100% is simulated according to an electrochemical model, taking a lithium cobaltate cell as an example. The process involves charging the lithium battery according to the product specification to a voltage of 4.2V at 0.5 C, then maintaining the voltage at 4.2V until the current drops to 0.05 C, and calculating the volume average concentration of positive lithium ions and the volume average concentration of negative lithium ions according to the electrochemical model.

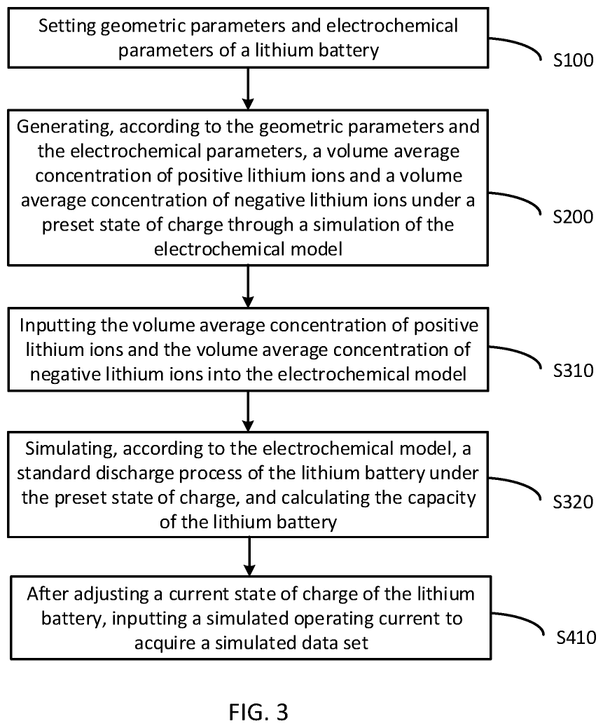

In an embodiment, as shown in FIG. 3, step S300 involves calculating, according to the volume average concentration of positive lithium ions and the volume average concentration of negative lithium ions under the preset state of charge, a capacity of the lithium battery through the simulation of the electrochemical model, including:

S310, inputting the volume average concentration of positive lithium ions and the volume average concentration of negative lithium ions into the electrochemical model.

S320, simulating, according to the electrochemical model, a standard discharge process of the lithium battery under the preset state of charge, and calculating the capacity of the lithium battery.

Exemplarily, after obtaining the volume average concentration of the positive lithium ions and the volume average concentration of the negative lithium ions at SOC=100%, the electrochemical model should be controlled to simulate a standard discharge process to obtain the capacity after modifying the model parameters, wherein the formula for calculating the lithium battery capacity is:

Q_{dis}=∫Idt;

wherein Q_{dis} is the discharge capacity of the lithium battery in the simulation process, which follows the standard cell discharge process, reducing the voltage of the lithium battery to 2.5V from 0.5 C.

In an embodiment, after calculating the discharge capacity Q_{dis} of the lithium battery during the simulation process with modified parameters, the capacity Q_{now} of the battery cell can be obtained by the ampere-hour integral method, and the SOH value of the battery can be further calculated using the following formula:

$$SOH = \frac{{Q}_{now}}{{Q}_{rated}} ;$$

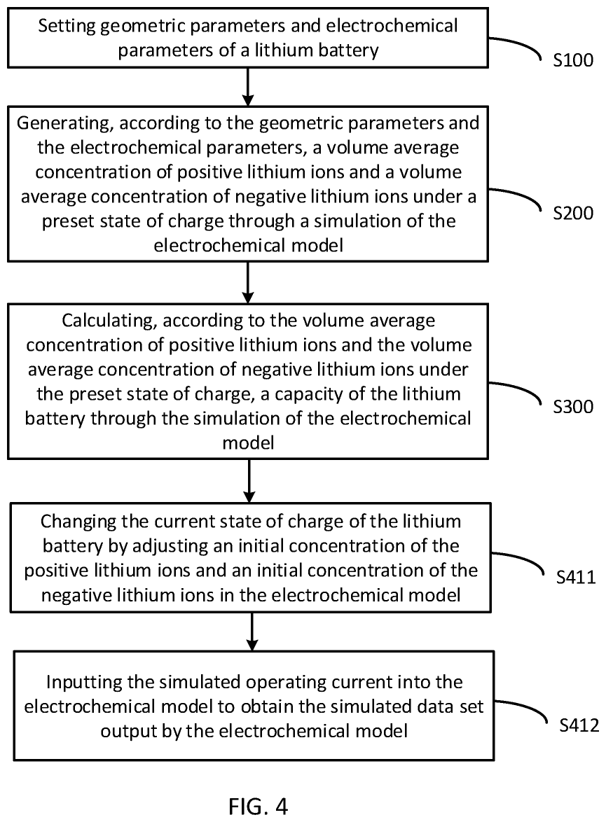

In an embodiment, as shown in FIG. 4, step S410 involves after adjusting a current state of charge of the lithium battery, inputting a simulated operating current to acquire a simulated data set, including:

S411, changing the current state of charge of the lithium battery by adjusting an initial concentration of the positive lithium ions and an initial concentration of the negative lithium ions in the electrochemical model.

Specifically, by adjusting the initial concentration of the positive lithium ions and the initial concentration of the negative lithium ions in the electrochemical model, the current state of charge of the lithium battery is changed to 80%, 60%, 40%, and the like, and different data sets are generated under the current parameters of the simulation of the electrochemical model.

S412, inputting the simulated operating current into the electrochemical model to obtain the simulated data set output by the electrochemical model.

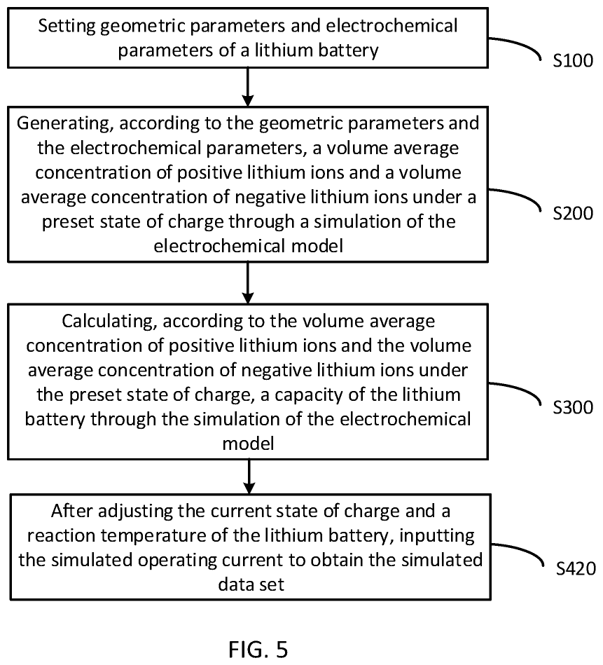

In an embodiment, as shown in FIG. 5, step S300 involves calculating, according to the volume average concentration of positive lithium ions and the volume average concentration of negative lithium ions under the preset state of charge, a capacity of the lithium battery through the simulation of the electrochemical model, after which the method further includes:

S420, after adjusting the current state of charge and a reaction temperature of the lithium battery, inputting the simulated operating current to obtain the simulated data set.

Specifically, by simultaneously adjusting the current state of charge and the reaction temperature of the lithium battery, it becomes possible to generate a richer data set under the same model parameters. Setting the simulated temperature allows for simulated data output by the battery cell under different SOCs and different temperatures to be obtained, improving the richness of the simulated data set.

In an embodiment, the simulated data set is an operating condition parameter of the lithium battery output by the electrochemical model, which may include, for instance, an OCV-SOC curve of the lithium battery.

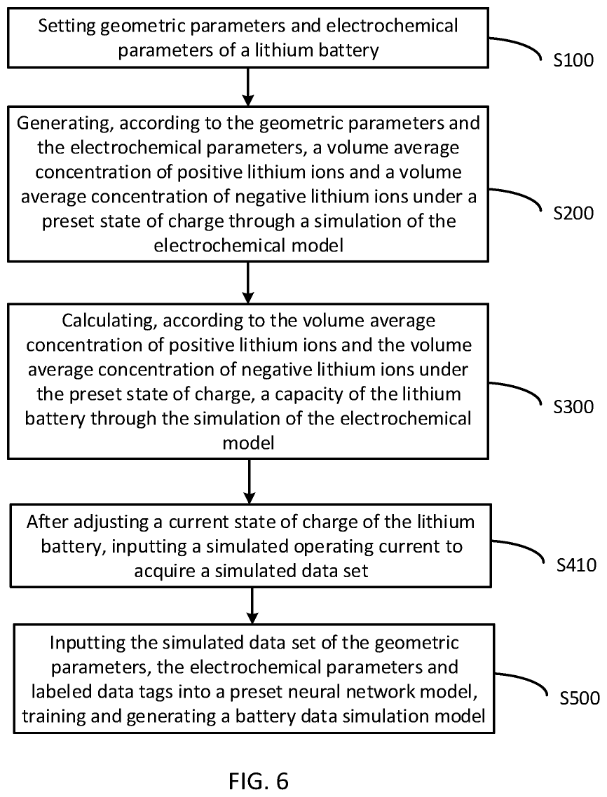

In an embodiment, as shown in FIG. 6, after step S410 or S420, the method further includes the steps of:

S500, inputting the simulated data set of the geometric parameters, the electrochemical parameters and labeled data tags into a preset neural network model, training and generating a battery data simulation model.

Specifically, the data tags include at least one parameter of the geometric parameters, the electrochemical parameters and the capacity of the lithium battery, and in this embodiment, by training the battery data simulation model, the generation process of the electrochemical model simulated data set is simplified, and the generation efficiency of the electrochemical model simulation data set is improved.

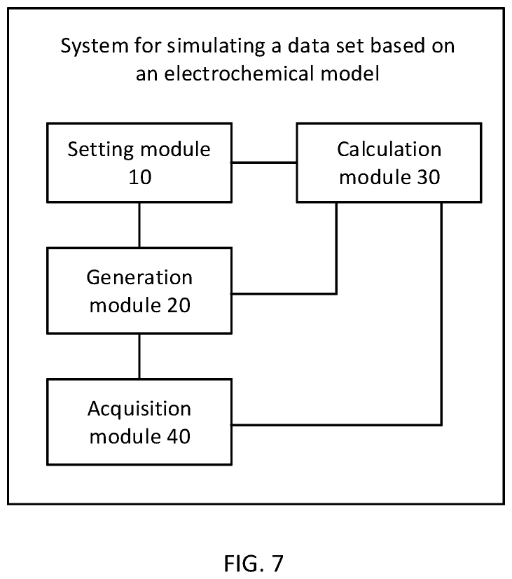

In an embodiment, as shown in FIG. 7, according to another aspect of the present invention, the present invention further provides a system for simulating a data set based on an electrochemical model, which includes a setting module 10, a generation module 20, a calculation module 30, and an acquisition module 40.

The setting module 10 is configured to set the geometric parameters and the electrochemical parameters of a lithium battery.

Specifically, by altering relevant parameters within an electrochemical model of the lithium battery, a plurality of operating condition data sets can be generated through simulation of the operating condition of the battery in the electrochemical model. For example, in setting geometric parameters, at least one of the parameters of surface area of the active material, thickness of the Ln negative electrode active material, thickness of the Lp positive electrode active material, and thickness of the Ls separator can be set, and in setting electrochemical parameters, at least one of the parameters of volume fraction of the active material, thickness of the SEI film, and solid phase conductivity can be set.

The generation module 20 is connected to the setting module 10, and configured to generate, according to the geometric parameters and the electrochemical parameters, a volume average concentration of positive lithium ions and a volume average concentration of negative lithium ions under a preset state of charge through a simulation of the electrochemical model

Specifically, when modifying the electrochemical parameters, the volume average concentration of positive lithium ions and the volume average concentration of negative lithium ions of the lithium battery will also change accordingly. Therefore, after adjusting the geometric parameters and the electrochemical parameters, the parameters of the electrochemical model need to be standardized to obtain the volume average concentration of positive lithium ions and the volume average concentration of negative lithium ions under the preset state of charge.

The calculation module 30 is connected to the setting module 10 and the generation module 20, and is configured to calculate, according to the volume average concentration of positive lithium ions and the volume average concentration of negative lithium ions under the preset state of charge, a capacity of the lithium battery through the simulation of the electrochemical model.

Specifically, after modifying the electrochemical model parameters, the capacity of the virtual lithium battery will change, so after obtaining the volume average concentration of positive lithium ions and the volume average concentration of negative lithium ions under the preset state of charge, simulation of the electrochemical model should be performed to obtain the capacity of the lithium battery after the model parameters are modified.

The acquisition module 40 is respectively connected to the calculating module 30 and the generating module 20, and is configured to, after adjusting a current state of charge of the lithium battery, input a simulated operating current to acquire a simulated data set.

Specifically, to ensure comprehensive battery simulation data, simulation data for the virtual lithium battery across different states of charge are obtained, serving as starting points for performing operating condition simulations.

According to the method for simulating a data set based on an electrochemical model provided by this embodiment, simulated data sets under different operating conditions are obtained based on the same electrochemical model through the steps of adjusting geometric parameters and electrochemical parameters of the lithium battery in the electrochemical model, standardizing parameters, calibrating capacity and adjusting the current state of charge of the lithium battery, reducing the difficulty in obtaining the simulated data of the electrochemical model.

In an embodiment, according to another aspect of the present invention, the present invention further provides a storage medium on which at least one instruction is stored, and the instruction is loaded and executed by a processor to implement operations of the method for simulating a data set based on an electrochemical model. For example, the storage medium may be a read-only memory (ROM), a random access memory (RAM), a compact disc read-only memory (CD-ROM), a magnetic tape, a floppy disk, an optical data storage device, and the like.

In the foregoing embodiments, the descriptions focus on specific aspects of the respective embodiments, and reference may be made to the related descriptions of other embodiments for parts that are not described or recited in detail in a certain embodiment.

Those skilled in the art will recognize that the various illustrative elements and steps described in connection with the embodiments disclosed herein may be implemented as electronic hardware or combinations of computer software and electronic hardware. Whether such functionality is implemented as hardware or software depends upon the particular application and design constraints imposed on the implementation. Skilled artisans may implement the described functionality in varying ways for each particular application, but such implementations should not be considered beyond the scope of the present application.

In the embodiments provided in the present application, it should be understood that the disclosed method, system, and storage medium for simulating a data set based on an electrochemical model may be implemented in other ways. For example, the above-described embodiments of a method, system, and storage medium for simulating a data set based on an electrochemical model are merely illustrative, and the division of the modules or units is only a logical functional division. In actual implementations, other divisions may be realized, for example, multiple units or modules may be combined or integrated into another system, or some features may be omitted or not executed. Furthermore, the communication links shown or discussed with respect to each other may be realized through some interfaces, systems, or unit communication links or integrated circuits, and may take various forms including electrical, mechanical or others.

The units described as separate parts may or may not be physically separate, and parts displayed as units may or may not be physical units, may be located in one position, or may be distributed across multiple network units. Depending on actual needs, some or all of the units can be selected to achieve the objectives of this embodiment.

In addition, functional units in the embodiments of the present application may be integrated into a single processing unit, or may each exist as separate physical units, or may be integrated as two or more units within a single unit. The integrated unit can be realized in the form of hardware or in the form of a software functional unit.

The foregoing description of the exemplary embodiments of the invention has been presented only for the purposes of illustration and description and is not intended to be exhaustive or to limit the invention to the precise forms disclosed. Many modifications and variations are possible in light of the above teaching. The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. A method for simulating a data set based on an electrochemical model, comprising:
setting geometric parameters and electrochemical parameters of a lithium battery;
generating, according to the geometric parameters and the electrochemical parameters, a volume average concentration of positive lithium ions and a volume average concentration of negative lithium ions under a preset state of charge through a simulation of the electrochemical model;
calculating, according to the volume average concentration of positive lithium ions and the volume average concentration of negative lithium ions under the preset state of charge, a capacity of the lithium battery through the simulation of the electrochemical model; and
after adjusting a current state of charge of the lithium battery, inputting a simulated operating current to acquire a simulated data set.

2. The method of claim 1, wherein the setting the geometric parameters and the electrochemical parameters of the lithium battery comprises:
setting the geometric parameters of the lithium battery, wherein the geometric parameters are battery structural parameters and are not influenced by an electrochemical reaction process after the lithium battery is manufactured; and
setting the electrochemical parameters according to adjustable requirements, wherein the electrochemical parameters are reaction coefficients in an electrochemical reaction of the battery and change along with progression of the electrochemical reaction process.

3. The method of claim 1, wherein the generating the volume average concentration of positive lithium ions and the volume average concentration of negative lithium ions under the preset state of charge through the simulation of the electrochemical model comprises:
inputting the geometric parameters and the electrochemical parameters into the electrochemical model; and
simulating, according to the electrochemical model, a standard charging process of the lithium battery under the preset state of charge, and calculating the volume average concentration of positive lithium ions and the volume average concentration of negative lithium ions.

4. The method of claim 1, wherein the calculating the capacity of the lithium battery through the simulation of the electrochemical model comprises:
inputting the volume average concentration of positive lithium ions and the volume average concentration of negative lithium ions into the electrochemical model; and
simulating, according to the electrochemical model, a standard discharge process of the lithium battery under the preset state of charge, and calculating the capacity of the lithium battery.

5. The method of claim 1, wherein the after adjusting the current state of charge of the lithium battery, inputting the simulated operating current and obtaining the simulated data set comprises:
changing the current state of charge of the lithium battery by adjusting an initial concentration of the positive lithium ions and an initial concentration of the negative lithium ions in the electrochemical model; and
inputting the simulated operating current into the electrochemical model to obtain the simulated data set output by the electrochemical model.

6. The method of claim 1, wherein the calculating the capacity of the lithium battery through the simulation of the electrochemical model further comprises:
after adjusting the current state of charge and a reaction temperature of the lithium battery, inputting the simulated operating current to obtain the simulated data set.

7. The method of claim 1, wherein the simulated data set includes an OCV-SOC curve of the lithium battery.

8. The method of claim 1, further comprising, after the obtaining the simulated data set:
inputting the simulated data set of the geometric parameters, the electrochemical parameters and labeled data tags into a preset neural network model, training and generating a battery data simulation model, wherein the data tags include at least one parameter of the geometric parameters, the electrochemical parameters and the capacity of the lithium battery.

9. A system for simulating a data set based on an electrochemical model, comprising:
a setting module, configured to set the geometric parameters and the electrochemical parameters of a lithium battery;
a generation module connected to the setting module, configured to generate, according to the geometric parameters and the electrochemical parameters, a volume average concentration of positive lithium ions and a volume average concentration of negative lithium ions under a preset state of charge through a simulation of the electrochemical model;
a calculation module respectively connected to the setting module and the generation module, configured to calculate, according to the volume average concentration of positive lithium ions and the volume average concentration of negative lithium ions under the preset state of charge, a capacity of the lithium battery through the simulation of the electrochemical model; and
an acquisition module respectively connected to the calculation module and the generation module, configured to, after adjusting a current state of charge of the lithium battery, input a simulated operating current to acquire a simulated data set.

10. A s non-transitory tangible computer-readable medium storing at least one instruction which, when executed by one or more processors, causes a system to perform the method for simulating a data set based on an electrochemical model according to claim 1.
